(19) United States
(12) Patent Application Publication
(10) Pub. No.: US 2022/0002433 A1
(43) Pub. Date: Jan. 6, 2022

(54) BRAIN TARGETED DRUG DELIVERY METHOD VIA SYNDECAN-3

(71) Applicant: PHARMACOIDEA Kft, Szeged (HU)

(72) Inventors: Anett HUDÁK, Szarvas (HU); Tamás LETOHA, Szeged (HU)

(73) Assignee: PHARMACOIDEA Kft, Szeged (HU)

(21) Appl. No.: 17/311,193

(22) PCT Filed: Dec. 5, 2019
(86) PCT No.: PCT/HU2019/000041

§ 371 Date: Jun. 4, 2021

(30) Foreign Application Priority Data
Dec. 5, 2018 (HU) HU-P1800420

(51) Int. Cl. C07K 16/28; A61K 39/395; A61K 47/68

(52) U.S. Cl. CPC C07K 16/2896; A61K 2039/505; A61K 47/6803; A61K 39/3955

(57) ABSTRACT

The invention relates to a method for brain targeted delivery of macromolecular protein or peptide ligands, including mono- or bispecific antibodies, nanobodies and active ingredients possessing specificity for 45 to 384 amino acid region of the SDC3 core protein into the brain, by a.) attaching the mono- or bispecific SDC3 antibody, nanobody, or mono or bispecific macromolecular ligands via systemic circulation to 45 to 384 amino acid region of SDC3 expressed on endothelial cells of the blood-brain-barrier, or b.) conjugating an active ingredient to an SDC3 specific mono- or bispecific antibody, nanobody, or mono or bispecific macromolecular ligands, and attaching the conjugate via systemic circulation to SDC3 expressed by endothelial cells of the blood-brain-barrier. Furtheron the invention relates to the use of a syndecan-3 target antibody, nanobody or macromolecular ligand, alone or in association with an active ingredient, as a therapeutic agent for treating neurodegenerative or metabolic disorders.

The present invention relates to a drug delivery method to the brain via syndecan-3.

Communication between cells and their environment, providing integrity for multicellular organisms, is based on cell surface receptor-ligand interactions. Due to their highly diverse three-dimensional heparan sulfate (HS) side chains, heparan sulfate proteoglycans (HSPGs), one of the most abundant families of cell surface membrane proteins, play an important role as receptors for large numbers of ligands (cytokines, growth factors, etc.). [Williams & Fuki 1998 (Curr. Opin. Lipidol 8 (5), 253-262); Bishop, Schuksz et al. 2007 (Nature 446 (7139) 1030-1037); Couchman 2010 (Annu. Rev. Cell Dev. Biol. 26, 89-114), Iozzo & Karamanos 2010 (FEBS J. 277 (19), 3863)].

There are two types of membrane HSPGs in mammalian cells: the glycosyl-phosphatidyl-inositol anchored glypicans predominantly expressed in the central nervous system (CNS) and the integral transmembrane protein syndecans (SDCs) that are more ubiquitous [Christianson & Belting 2014 (Matrix Biol. 35: 51-55)].

There are four syndecan (SDC) isoforms: syndecan-1 (SDC1) expressed on epithelial and plasma cells, syndecan-2 (SDC2) on endothelial cells and fibroblasts, syndecan-3 (SDC3) on neurons and the syndecan-4 (SDC4) that is universally expressed [Tkachenko & Rhodes 2005 (Circ. Res. 96 (5), 488-500); Afratis, Nikitovic et al. 2017 (FEBS. J. 284 (1), 27-41)] (FIG. 1).

Due to their diverse heparan sulfate chains, SDCs are capable of binding many endogenous and exogenous ligands, including, but not limited to growth factors, cytokines and parasites [Tkachenko et al. 2005 (Circ. Res. 96 (5), 488-500)]. In addition to their role in signal transduction, SDCs are also involved in endocytosis: binding of certain macromolecular ligands (proteins, peptides, viruses and bacteria) to SDCs induces the cellular internalization of the ligand-SDC complex [Christianson and Belting 2014 (Matrix Biol. 35, 51-55)]. Literature data claims that SDC-dependent cellular entry of ligands requires attachment to SDCs' HS side chains. According to Wittrup et al., binding to SDC HS side chains triggers SDC-dependent uptake by an isoform-specific manner: cellular internalization of a single-chain variable fragment (scFv) anti-HS antibody is stimulated by SDC2, while inhibited by SDC3 [Wittrup et al. 2009 (J Biol Chem, 284 (47), 32959-67)].

SDC3 (neuronal- or N-syndecan) is a 442 amino acid long transmembrane protein predominantly expressed in neurons [Carey et al. 1992 (J Cell Biol. 117 (1): 191-201); Berndt et al. 2001 (J Cell Biochem. 82 (2): 246-59)]. SDC3 has four conserved glycosaminoglycan binding sites at the N-terminus and contains unique threonine-rich and mucin-like regions close to the membrane [Chernousov, & Carey 1993 (J Biol Chem 268, 16810-16814); Asundi & Carey 1995 (J Biol Chem 270, 26404-26410); De Rossi & Whiteford 2013 (F1000Research 2, 270)]. The HS region of SDC3 serves as a binding site for several growth factors, including AgRP (Agouti-related protein), HB-GAM (heparin-binding growth-associated molecule), GDNF (glial cell line-derived growth factor), NRTN (neurturin), artemin and NOTCH [Bespalov, Sidorova et al. 2011 (J Cell Biol. 192 (1): 153-169); Creemers, Pritchard et al. 2006 (Endocrinology. 147 (4): 1621-1631); Nolo, Kaksonen et al. 1995 (Neurosci Lett. 191 (1-2): 39-42); Pisconti, Cornelison et al. 2010 (J Cell Biol. 190 (3): 427-441); Reizes, Benoit et al. 2003 (Ann NY Acad Sci. 994: 66-73)]. SDC3 also plays an important role in viral infections: as a receptor for the human immunodeficiency virus type 1 (HIV-1), SDC3 mediates HIV-1 transmission to T cells [de Witte, Bobardt et al. 2007 (Nat. Acad. Sci. USA 10449, 19464-19469)]. Moreover, SDC3 plays a role in the regulation of memory and metabolism [Kaksonen, Pavlov et al. 2002 (Mol. Cell. Neurosci. 21 (1), 158-172); Strader, Reizes et al. 2004 (J. Clin. Invest. 114 (9 (1354-1360); Reizes, Benoit et al. 2008 (Int. J. Biochem. Cell. Biol. 40 (1), 28-45)]. Expression of SDC3 is significantly increased in the brains of Alzheimer's disease patients [Liu, Zhao et al. 2016 (Sci. Trans. Med. 8 (332), 332-344)].

SDC3 is known to be present in the cerebral blood vessels of the blood-brain-barrier (BBB) and facilitates transendothelial migration of monocytes into the brain [Floris, van den Born et al. 2003 (J. Neuropathol. Exp. Neurol. 62 (7), 780-790)]. SDC3-mediated monocyte migration through the BBB also requires the attachment of monocytes to the HS chains of SDC3. Other literature data also highlights the role of HS side chains of SDC3 in transmigration of HIV-1 through the BBB. According to these data, migration of HIV-1 through the BBB is mediated by electrostatic interactions between basic residues of HIV-1's gp120 glycoprotein and polyanionic HS chains of SDC3 [Bobardt et al. 2004 (J Virol. 78 (12): 6567-84.)].

To review the evidence on the role of SDC3 in BBB transport of macromolecules, we measured the SDC3 expression of hCMEC/D3 endothelial cells isolated from human temporal lobe microvessels with flow cytometry by using allophycocyanin (APC) labeled human SDC3 antibody, a polyclonal goat IgG raised against GIn48-Lys383 region of recombinant human SDC3 (R&D Systems, cat. no. FAB3539A). In these studies, hCMEC/D3 endothelial cells showed high SDC3 expression (FIG. 2A). Removal of the SDC heparan sulfate side chains by heparinase (HPase) did not affect the binding of the SDC3 antibody (while significantly blocking the attachment of anti-HS monoclonal 10E4 antibody), indicating that the applied SDC3 antibody was specific for the GIn48 and Lys383 region of the SDC3 core protein but not for its HS chains (FIG. 2B).

After incubating the human hCMEC/D3 endothelial cells for 3h at 37° C. with the SDC3 antibody specific for the GIn48 and Lys383 region of the SDC3 core protein (R&D Systems, Cat. No. FAB3539A, concentration 1.25 μg/mL), the fluorescently labeled SDC3 antibody appeared inside the cells (FIG. 3A). Internalization of the SDC3 antibody was not affected by the removal of HS side chains with heparinase (FIG. 3B). These results contradict literature claims and reveal that efficient intracellular entry via SDC3 can be facilitated by specific attachment to GIn48-Lys383 region of the SDC3 core protein.

In in vivo studies, female and male C57BL/6 (wild-type [WT]) and APPSWE-Tau mice, at least 6 months of age (Taconic Biosciences) were injected intravenously at a dose of 1 mg/kg with SDC3 antibodies (monoclonal rat IgG2A or polyclonal goat IgG, both raised against Ala45-Glu384 of NSO mouse myeloma cell line-derived mouse SDC3 [R&D Systems, cat. no. MAB2734 and AF2734]). Control mice were treated with 200 μL PBS in the same manner. The WT C57BL/6 and APPSWE-Tau mice were randomly assigned to antibody-treated and control (i.e. PBS-treated) groups. One hour after treatment, the mice were anesthetized with Avertin and transcardially perfused with ice-cold PBS (2 ml/min for 8 min). After perfusion, the brain was isolated, dissected frontally, and frozen in dry ice for further Western blot and microscopic examination. For Western blot, brain samples were homogenized in lysis buffer (QIAGEN) in 1% NP-40/PBS in Complete Mini EDTA-free protease inhibitor cocktail (Roche) and tissue lysates were run on 15% gel. For detecting the SDC3 antibodies in brain samples of animals treated with SDC3 antibodies (either monoclonal or polyclonal), a secondary anti-rat or anti-goat IgGs labeled with Alexa Fluor 647 were used (FIGS. 4A and B). For immunohistochemistry, mouse brain samples were fixed for 18 h in 4% paraformaldehyde, and 30 μm thick sagittal sections were sliced with a Leica microtome (n=6 mice per group). The fluorescence distribution was examined using a BioTek Cytation 3 Cell Imaging Multi-Mode Reader. Examination of the resulting brain sections revealed that the SDC3 antibodies (either monoclonal or polyclonal) appeared in the brain of antibody-treated mice and enriched in the neurons (FIGS. 4C and D).

Next we investigated the effect of the SDC3 core protein specifc antibody on the binding of beta-amyloid peptide (Aβ)—the main component of the Alzheimer's disease-related amyloid plaques—to neurons. It has been well documented, that Aβ attaches to the HS chains of HSPGs and this attachment triggers fibrillation leading to plaque formation [Fukuchi et al. 1998. (Frontiers in bioscience 3, d327-337); Small et al. 1996 (Annals of the New York Academy of Sciences 777, 316-321); Wesen et al. 2017 (Sci. Reports 7, 2021); Bruinsma et al. 2010 (Acta neuropathologica 119, 211-220)]. Considering the evidence on the HS-dependent attachment of Aβ to neurons, we were interested whether an SDC3 antibody specific for the core protein, but not the HS chains of the SDC3 ectodomain would interefere with binding of Aβ to neurons. SH-SY5Y cells expressing SDC3 were treated with 5 μM fluorescently labeled (FITC) Aβ1-42 at 37° C. for 18 hours with or without SDC3 antibody at a concentration of 1.25 μg/mL human (either monoclonal rat IgG2A or polyclonal goat IgG, manufactured by R&D systems, cat. no. MAB35391 and FAB3539A, respectively). Following incubation, cells were assayed with a FACScan flow cytometer (Becton Dickinson FACScan). Studies showed that the SDC3 antibodies—either monoclonal or polyclonal—specific for region GIn48-Lys383 region of human SDC3, significantly (˜40%, p<0.05) inhibited the binding of Aβ1-42 to SH-SY5Y cells (FIG. 5A). Flow cytometry results were also confirmed with scanning electron microscopy, showing reduced number of Aβ1-42 fibrils on cells pretreated with SDC3 antibodies (FIG. 5B).

The in vivo effect of the SDC3 antibody on plaque formation was then investigated in female and male APPSWE-Tau mice at least 6 months of age (Taconic Biosciences). APPSWE-Tau mice (Taconic Biosciences) were injected intraperitoneally with the SDC3 antibody (monoclonal rat IgG2A [mAB] or polyclonal goat IgG [pAB]—all R&D Systems, cat. no. MAB2734 and AF2734), dissolved in 200 μL sterile PBS) at a dose of 1 mg/kg. Control mice were treated with 200 μL PBS in the same manner. APPSWE-Tau mice were randomly assigned to antibody-treated and control groups (n=4 animals per group). Treatment was given twice weekly for 3 months (12 weeks). After 3 months of treatment, mice were anesthetized with Avertin, transcardially perfused with ice-cold PBS (2 ml/min, 8 min). After perfusion, the brain was isolated, dissected frontally, and frozen in dry ice for further Western blot and microscopic examination. For immunohistochemistry, mouse brain samples were fixed for 18 h in 4% paraformaldehyde, and 30 μm thick sagittal sections were sliced with a Leica microtome (n=3 mice per group). Sections were stained with Thioflavin T (ThT). The number of plaques was measured by fluorescence distribution using BioTek Cytation 3 Cell Imaging Multi-Mode Reader. Differences between experimental groups were evaluated by using one-way analysis of variance (ANOVA). Mouse brain samples after 3 months of treatment clearly showed that SDC3 antibody (either monoclonal or polyclonal) treatment significantly (p<0.05) reduced the number of amyloid plaques in the brain (FIGS. 6A and B).

The present invention is based on the surprising finding that

a.) mono- or bispecific macromolecular protein or peptide ligands, including mono- or bispecific antibodies, nanobodies, possessing specificity for the 45 to 384 amino acid region of the SDC3 core protein, but not its HS chains, are able to enter the brain via SDC3-mediated transport, and
b.) by administering the SDC3 antibody or macromolecular ligands into the systemic circulation, the SDC3 antibody or macromolecular ligand, possessing specificity for the 45 to 384 amino acid region of the SDC3 core protein, is ferried across the blood-brain-barrier (BBB)—via an SDC3-mediated transport process—into the brain, where it binds SDC3 and inhibits SDC3 dependent cellular processes related to metabolic disorders (obesity, etc.) and neurodegeneration (Alzheimer's/Parkinson's disease).

As SDC3 macromolecular ligands, antibodies, peptides, proteins, or other molecules that are naturally occurring or synthetically produced (hereinafter referred to as “active ingredients” or “active substance”)) can be used. Those skilled in the art will be able to select these active ingredients from the state of the art.

Accordingly the invention relates to syndecan-3 target antibody, nanobody or macromolecular ligand possessing specificity for 45 to 384 amino acid region of the syndecane-3 core protein alone or in association with an active ingredient for use in the treatment of neurodegenerative or metabolic disorders.

Furtheron the invention relates to the use of a syndecan-3 target antibody, nanobody or macromolecular ligand possessing specificity for 45 to 384 amino acid region of the syndecane-3 core protein alone or in association with an active ingredient, as a therapeutic agent for treating neurodegenerative or metabolic disorders.

A further embodiment of the invention is a method for brain targeting of a syndecan-3 antibody, nanobody or macromolecular ligand possessing specificity for 45 to 384 amino acid region of the syndecane-3 core protein alone or in association with an active ingredient capable of specifically binding to syndecan-3 expressed on endothelial cells of the blood-brain-barrier, characterized by delivering said antibody, nanobody or macromolecular ligand into the brain.

As antibody, nanobody or macromolecular ligand macromolecular protein or peptid ligands, including mono- or bispecific antibodies, nanobodies may be used.

According to the invention the delivery is carried out, by
a.) attaching the mono- or bispecific syndecan-3 antibody, nanobody, or mono or bispecific macromolecular ligands via systemic circulation to 45 to 384 amino acid region of syndecan-3 expressed on endothelial cells of the blood-brain-barrier, or
b.) conjugating an active ingredient to a syndecan-3 specific mono- or bispecific antibody, nanobody, or mono or bispecific macromolecular ligands, and attaching the conjugate via systemic circulation to syndecan-3 expressed by endothelial cells of the blood-brain-barrier.

Preferrable a bispecific antibody, nanobody or ligand more preferable a bispecific active ingredient is used.

When using a bispecific antibody, nanobody, or macromolecular ligand, translocation through the BBB is provided by the specificity of the antibody, nanobody, or macromolecular ligand for the 45 to 384 amino acid region of SDC3, while the other specificity of the antibody, nanobody, or macromolecular ligand for another CNS target provides attachment to the other targets in the brain.

SDC3 targeting antibody, nanobody, or macromolecular ligand, alone or linked to other active substance, after passing the BBB, binds to endogenous SDC3 expressed on neurons and interfere with SDC3-dependent cellular process of neurodegenerative or metabolic pathways, hence could be used as a therapeutic agent in neurodegenerative or metabolic disorders.

The syndecan-3-mono or bispecific antibody, nanobody, or mono or bispecific macromolecular ligand, or the active ingredient attached thereto, is preferably delivered to the brain by conjugation to a carrier (liposome, nanocarrier, peptide).

### EXPLANATION OF THE FIGURES

FIG. 1. Schematic representation of SDCs [Letoha et al. 2010 (Biochim Biophys Acta. December; 1798(12):2258-65)].

FIG. 2. SDC expression of hCMEC/D3 endothelial cells.

FIG. 3. Cellular entry of the SDC3 antibody.

FIG. 4. In vivo delivery of the SDC3 antibody into the brain.

FIG. 5. The SDC3 antibody inhibits cellular attachment and uptake of Aβ1-42.

FIG. 6. The SDC3 antibody inhibits plaque formation.

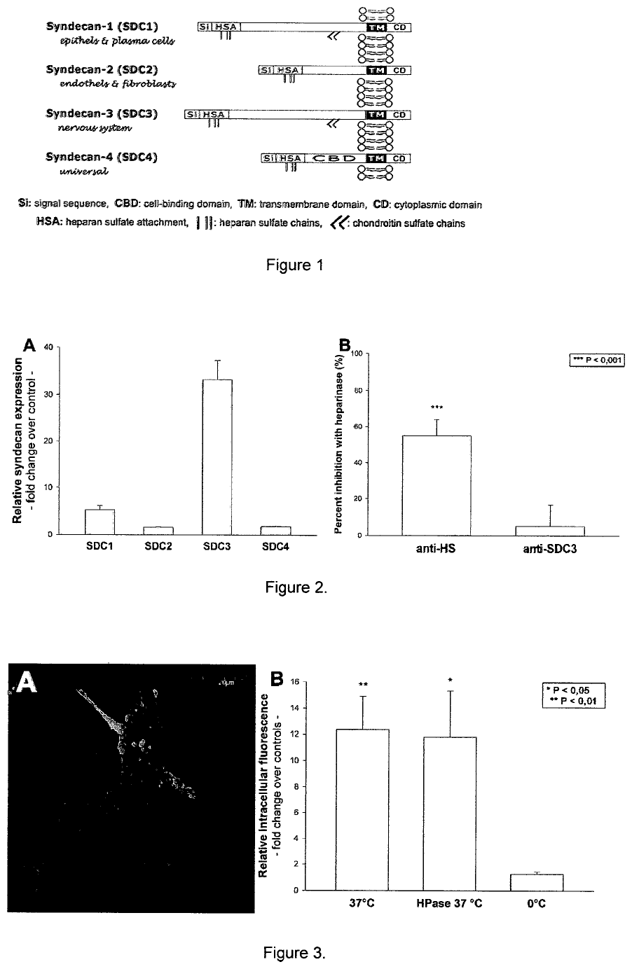

More details of the invention are described in the examples without to restrict the invention to the examples.

### EXAMPLE 1 SDC EXPRESSION OF HCMEC/D3 ENDOTHELIAL CELLS

FIG. 2A: 3×10⁵ hCMEC/D3 cells were incubated with APC-labeled antibodies specific for each SDC isoforms (R&D Systems, cat. No. FAB2780A, FAB2965A, FAB3539A, FAB29181A) according to the manufacturer's protocols. SDC expression was then measured by flow cytometry using a FACScan (Becton Dickinson). Detected fluorescence of antibody-treated cells were normalized to untreated controls as standards. The bars represent mean±SEM of three independent experiments.

FIG. 2B: Attachment of the SDC3 antibody specific for human GIn48-Lys383 region of the SDC3 core protein was also analyzed. hCMEC/D3 cells were treated with 12.5 U/mI of heparinase I and III blend at 37° C. for 3 h before being subjected SDC3 antibody treatment. The effectiveness of HPase treatments was verified by also measuring the HS expression, using anti-human HS antibody (10E4 epitope [Amsbio]) and FITC-labeled secondary IgG (Sigma) according to the manufacturers' protocols. The effect of heparinase was expressed as percent inhibition. The bars represent mean±SEM of three independent experiments. Statistical significance vs controls was assessed by analysis of variance (ANOVA). *p<0.05 vs controls; **p<0.01 vs controls; ***p<0.001 vs controls.

### EXAMPLE. 2. CELLULAR ENTRY OF THE SDC3 ANTIBODY

hCMEC/D3 cells were treated with the APC-labeled SDC3 antibody at 37° C. (or 0° C.) at a concentration of 1.25 μg/mL for 3 h. Following 3 hours of antibody treatment, cellular uptake was studied either with confocal microscopy (A) or flow cytometry (B). Before the flow cytometry measurements, the cells were trypsinized for 15 minutes to remove exogenously adherent antibody, allowing the flow cytometer to measure only intracellular fluorescence

FIG. 3A: Confocal microscope images of hCMEC/D3 endothelial cells treated with the SDC3 antibody at 37° C.

FIG. 3B: Flow cytometry results of hCMEC/D3 cells treated with the APC-labeled SDC3 antibody at 37° C. or 0° C. SDC3 antibody uptake was also analyzed on cells pretreated with 12.5 U/ml of heparinase I and III blend (Sigma) at 37° C. for three hours before being subjected SDC3 antibody treatment. The bars represent mean±SEM of three independent experiments. Statistical significance vs controls was assessed by analysis of variance (ANOVA). *p<0.05 vs controls; **p<0.01 vs controls; ***p<0.001 vs controls.

### EXAMPLE 3. IN VIVO DELIVERY OF THE SDC3 ANTIBODY INTO THE BRAIN

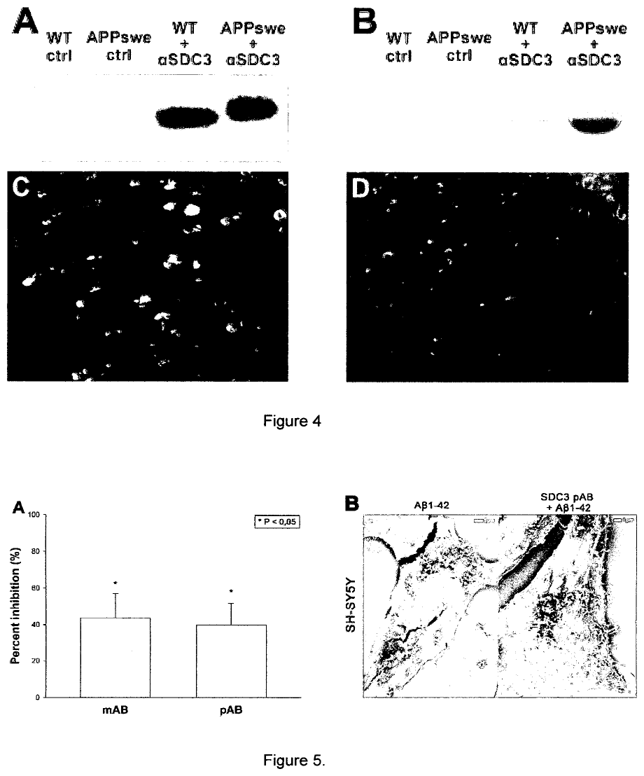

FIGS. 4A-B: Western-blot images showing the presence of the SDC3 antibody in brain extract of wild-type (WT) and APPswe mice treated with SDC3 antibody. SDC3 antibody was detected by Uvitec's ALLIANCE Q9 ADVANCED imaging platform.

FIGS. 4C-D: Microscopic images of WT and APPswe mice treated with SDC3 antibody.

### EXAMPLE 4. THE SDC3 ANTIBODY INHIBITS CELLULAR ATTACHMENT AND UPTAKE OF Aβ1-42

SH-SY5Y cells were treated with 5 μM fluorescently labeled (FITC) Aβ1-42 at 37° C. for 18 hours with or without SDC3 antibody at a concentration of 1.25 μg/mL human (monoclonal rat IgG2A [mAB] or polyclonal goat IgG [pAB], all manufactured by R&D systems, cat. no. MAB35391 and FAB3539A, respectively). Following incubation, cellular fluorescence was measured with a FACScan flow cytometer (Becton Dickinson FACScan).

FIG. 5A: Results of the flow cytometry measurements. The effect of SDC3 antibodies (either mAB or pAB) on cellular attachment and uptake of Aβ1-42 was expressed as percent inhibition. The bars represent mean±SEM of three independent experiments. Statistical significance vs cells treated with Aβ1-42 only (i.e. controls) was assessed by analysis of variance (ANOVA). *p<0.05 vs controls.

FIG. 5B: Flow cytometry results were also confirmed using a scanning electron microscope (JEOL JSM-7100F/LV), hence visualizing the surface SH-SY5Y cells were treated with 5 μM fluorescently labeled (FITC) Aβ1-42 at 37° C. for 18 hours with or without SDC3 antibody at a concentration of 1.25 μg/mL.

### EXAMPLE 5. THE SDC3 ANTIBODY INHIBITS PLAQUE FORMATION

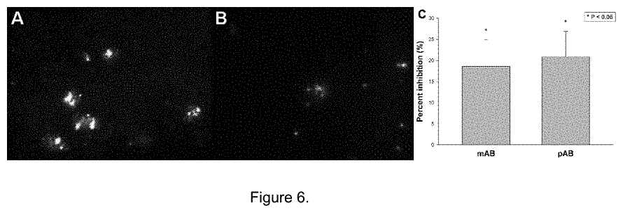

FIGS. 6A-B: Representative brain section of APPSWE-Tau mice untreated (A) or treated (B) with SDC3 antibody. Brain sections were stained with Thioflavin T (ThT). The number of plaques was measured by fluorescence distribution using BioTek Cytation 3 Cell Imaging

Multi-Mode Reader.

FIG. 6C: The effect of SDC3 antibodies (either mAB or pAB) on plaque formation was expressed as percent inhibition. The bars represent mean±SEM of four animals per group. Differences between experimental groups were evaluated by using one-way analysis of variance (ANOVA). Statistical significance vs untreated APPSWE-Tau mice (i.e. controls) was assessed by analysis of variance (ANOVA). *p<0.05 vs controls.

1-5. (canceled)

6. Syndecan-3 target antibody, nanobody or macromolecular ligand possessing specificity for 45 to 384 amino acid region of the syndecane-3 core protein alone or in association with an active ingredient for use in the treatment of neurodegenerative or metabolic disorders.

7. Use of a syndecan-3 target antibody, nanobody or macromolecular ligand possessing specificity for 45 to 384 amino acid region of the syndecane-3 core protein alone or in association with an active ingredient, as a therapeutic agent for treating neurodegenerative or metabolic disorders.

8. A method for brain targeting of a syndecan-3 antibody, nanobody or macromolecular ligand possessing specificity for 45 to 384 amino acid region of the syndecane-3 core protein alone or in association with an active ingredient capable of specifically binding to syndecan-3 expressed on endothelial cells of the blood-brain-barrier, characterized by delivering said antibody, nanobody or macromolecular ligand into the brain.

9. The method according to claim 8, characterized by that as antibody, nanobody or macromolecular ligand macromolecular protein or peptid ligands, including mono- or bispecific antibodies, nanobodies are used.

10. The method according to claim 8, characterized by that the delivery is carried out, by
a. attaching the mono- or bispecific syndecan-3 antibody, nanobody, or mono or bispecific macromolecular ligands via systemic circulation to 45 to 384 amino acid region of syndecan-3 expressed on endothelial cells of the blood-brain-barrier, or
b. conjugating an active ingredient to a syndecan-3 specific mono- or bispecific antibody, nanobody, or mono or bispecific macromolecular ligands, and attaching the conjugate via systemic circulation to syndecan-3 expressed by endothelial cells of the blood-brain-barrier.

11. The method of claim 8, wherein a bispecific antibody, nanobody or ligand is used.

12. The method of claim 8, wherein a bispecific active ingredient is used.

13. The method of claim 8, wherein the syndecan 3 mono or bispecific antibody, nanobody, or mono or bispecific macromolecular ligand or active ingredient is conjugated to a carrier (liposome, nanocarrier, peptide).

14. The method according to claim 9, characterized by that the delivery is carried out, by
a. attaching the mono- or bispecific syndecan-3 antibody, nanobody, or mono or bispecific macromolecular ligands via systemic circulation to 45 to 384 amino acid region of syndecan-3 expressed on endothelial cells of the blood-brain-barrier, or
b. conjugating an active ingredient to a syndecan-3 specific mono- or bispecific antibody, nanobody, or mono or bispecific macromolecular ligands, and attaching the conjugate via systemic circulation to syndecan-3 expressed by endothelial cells of the blood-brain-barrier.

15. The method of claim 9, wherein a bispecific antibody, nanobody or ligand is used.

16. The method of claim 10, wherein a bispecific antibody, nanobody or ligand is used.

17. The method of claim 9, wherein a bispecific active ingredient is used.

18. The method of claim 10, wherein a bispecific active ingredient is used.

19. The method of claim 9, wherein the syndecan 3 mono or bispecific antibody, nanobody, or mono or bispecific macromolecular ligand or active ingredient is conjugated to a carrier (liposome, nanocarrier, peptide).

20. The method of claim 10, wherein the syndecan 3 mono or bispecific antibody, nanobody, or mono or bispecific macromolecular ligand or active ingredient is conjugated to a carrier (liposome, nanocarrier, peptide).
